(19) United States
(12) Patent Application Publication
(10) Pub. No.: US 2011/0287982 A1
(43) Pub. Date: Nov. 24, 2011

(54) CELL CULTURE DEVICE

(76) Inventor: Luc Stoppini, Geneva (CH)

(21) Appl. No.: 13/060,062

(22) PCT Filed: Aug. 21, 2009
(86) PCT No.: PCT/IB2009/006725

§ 371 Date: Aug. 11, 2011

(30) Foreign Application Priority Data
Aug. 22, 2008 (GB) 0815427.0

(51) Int. Cl. C40B 60/14; C12N 5/071; C12Q 1/02; C12M 1/12

(52) U.S. Cl. 506/40; 435/297.1; 435/325; 435/29

(57) ABSTRACT

The invention relates to the field of cell and tissue culture. In particular, the invention relates to devices for cell culture. A device for cell culture, according to the invention comprises (i) a medium conduit having one open end and one end closed by a porous membrane fused across it; and (ii) a frame holding the medium conduit in a substantially vertical orientation; wherein the medium conduit is adapted to permit retention by capillarity of a sufficient volume of liquid culture medium in the medium conduit to contact the surface of the porous membrane and thus supply nutrients to cells that may be grown on the porous membrane, characterised in that the surface of the membrane contralateral to the surface of said porous membrane sealed to said medium conduit comprises a hydrophobic barrier adapted to contain the culture.

The invention relates to the field of cell and tissue culture. In particular, the invention relates to devices for cell culture.

Tissue culture is the ex vivo maintenance of cells that originated from an organ or tissue of an animal or plant organism. Methods of tissue culture have been developed and improved over many decades.

A major advance in the field of tissue culture has been the introduction of organotypic culture methods for organ and tissue slices. Thin (50-500 μm) slices of an animal organ are cultured under conditions in which the slices retain the cellular composition, morphology and the physiological properties of the source organ. The conditions in which the organ slices are cultured are critical to achieve organotypic culture. The organ slices are cultured on the upper surface of a porous membrane and supplied with nutrient from the lower surface of said porous membrane such that the organ slice is not fully immersed but is covered only by a thin film of culture medium (Stoppini L. et al, 1991).

Gas transfer to the slice, both for the uptake of oxygen and the removal of carbon dioxide, is much more efficient than when the slice is fully immersed in culture medium according to the methods of explant culture. In addition, organotypic slice culture does not suffer from the disadvantages associated with explant-based culture, e.g. the limited period of time before the cells undergo a form of cell death called senescence and the propensity of explant-based animal cell primary culture cells to lose many of the characteristics that are typical of cells in the source organ in vivo.

A further advance in the field of tissue culture has been the introduction of culture methods which allow the production of an organotypic culture from dissociated cells or from small aggregates of cells (microexplants or explants) on a membrane by compacting them. The contralateral side of the membrane is supplied with nutrients in accordance with the teaching of Stoppini L. et al, 1991. Methods of producing an organotypic culture using dissociated cells or microexplants instead of organ slices are described in WO2006/136953. A device suitable for carrying out such methods is described in WO2006/134432. The device allows culture of cells at the air liquid interface and allows for the preparation and maintenance of cell cultures and facilitated medium change without subjecting the cell culture to unnecessary stress. Furthermore, the device is designed to facilitate high-throughput production and screening of the cell cultures.

There remains a need for improved devices for cell culture and the inventors of the present application have succeeded in making a number of surprising improvements to the device described in WO2006/134432.

### SUMMARY OF THE INVENTION

According to a first aspect of the invention, there is provided a device for cell culture, said device comprising:
(i) a medium conduit having one open end and one end closed by a porous membrane fused across it; and
(ii) a frame holding the medium conduit in a substantially vertical orientation;

wherein the medium conduit is adapted to permit retention by capillarity of a sufficient volume of liquid culture medium in the medium conduit to contact the surface of the porous membrane and thus supply nutrients to cells that may be grown on the porous membrane, characterised in that the surface of the membrane contralateral to the surface of said porous membrane sealed to said medium conduit comprises a hydrophobic barrier adapted to contain the culture.

The presence of a hydrophobic barrier adapted to contain the culture on the membrane confers a number of advantages over the devices known in the prior art. For example, cell cultures grown using the device described in WO2006/134432 are grown on the upperside of a porous membrane at the air-liquid interface. However, the device in WO2006/134432 does not provide a means for containing the growth of the culture, i.e. the edges of the membrane are not designed to restrict cell growth. Therefore, proliferation of the culture can result in it growing beyond the edges of the membrane and onto the device itself. This prevents the correct supply of medium to the culture and makes further handling of culture difficult.

The inventors have surprisingly found that a hydrophobic barrier can be used to delimit the boundaries of the cell culture and prevent “over growth” of the culture beyond the membrane and onto the device.

Cultures grown on the device described in WO2006/134432 grow at the air-liquid interface. The air-liquid interface is formed due to the porous nature of the membrane and the gravitational force exerted on the liquid medium surrounding the cell culture. Gravity acts to draw any excess liquid medium contained in the cell culture through the porous membrane and away from the cell culture. At the same time, surface tension in the liquid medium means that not all of the medium is drawn away, but instead a layer of medium is left coating the cell culture. The growth of cell cultures at the air-liquid interface is advantageous to the cell cultures.

If no barrier is placed around the culture, once the culture has grown to the edge of the membrane, then capillarity may cause the medium to be drawn over the edges of the membrane. In this case, the culture will no longer be at the air-liquid interface, but will instead by submerged in the excess medium being drawn onto the upperside of the membrane. The culture will, in such a case, be flooded.

The inventors have surprisingly realised that the presence of a hydrophobic barrier in the device of the present invention prevents such flooding.

The inventors have found that the prevention of flooding is more effective when the height that the hydrophobic barrier projects above the membrane is below 100 μm, e.g. about 1, 2, 5, 10, 15, 20, 25, 30, 35, 40, 50, 60, 70, 80, 90 or 100 μm. More preferably, the hydrophobic barrier projects no further than 50 μm above the surface of the membrane.

As well as preventing flooding, the hydrophobic barrier allows the boundaries of the cell culture to be controlled. Therefore, the shape and size of the cell culture can be altered as desired. The barrier can be of any shape, for example it may be circular, elliptical, triangular or square. The barrier can also be of more complex shapes such as a dumbbell. FIG. 2 shows a number of different shapes which may be used for the hydrophobic barrier.

The shape of the barrier may be chosen based on the type of cells being cultured. For example, it may be desirable to grow neuronal cells within dumbbell shaped hydrophobic barriers, while it may be desirable to grow pancreas or liver cells within circular shaped hydrophobic barriers.

The area contained within the hydrophobic barrier can also be altered. If the barrier is circular, then the radius will usually be in the range of 0.5 mm to 5.0 mm, e.g. 0.5, 0.75, 1.0, 2.0, 3.0, 4.0 or 5.0 mm. If the hydrophobic barrier is any other shape, then the area contained within the hydrophobic barrier will usually be in the range of 0.5 mm² to 80 mm², e.g. 0.5, 1.0, 2.0, 5.0, 10, 15, 20, 25, 30, 35, 40, 45, 50, 60, 70, 80 mm².

The use of the hydrophobic barrier also allows culture conditions to be changed when the culture reaches a specific predetermined shape or size. For example, a stem cell culture may be grown within a hydrophobic barrier in the device of the invention. The medium used to sustain the culture can be controlled so that the culture is allowed to proliferate, e.g. the inclusion of embryonic growth factor (EGF) or foetal growth factor (FGF). Proliferation can be continued until the cell culture fills the area within the hydrophobic barrier. At this stage, the medium can altered, e.g. by removing EGF or FGF, and the culture can be allowed to differentiate. Such control means that cell cultures of precise size and shape can be consistently generated. The generation of multiple cultures in this way improves the repeatability of experiments conducted using the cell cultures generated with the device of the invention.

The hydrophobic barrier also allows the precise location of the cell culture to be known. This increases the efficiency with which the cultures present on the device can be located. The efficiency can be further increased by using a hydrophobic barrier which is coloured in such a way so that it contrasts with the colour of the porous membrane. For example, the hydrophobic barrier may be red, blue, green, black, grey, yellow, orange, or any shade of these colours.

Furthermore, the hydrophobic barrier also confers advantages to the automation of the cell culture. It is currently known to use robotic arms to apply cell cultures to multi-well plates. However, as it is important to avoid damage of the membrane caused by the pipette, the pipette tip is not allowed to advance into contact with the membrane. This in turn can cause a slight variation in the location that the initial culture is pipetted onto the membrane. Therefore, subsequent automated procedures become increasingly difficult as the exact location of the culture is not known.

The device of the invention overcomes this disadvantage by allowing the location of the cell culture to be known precisely, i.e. it is always within the boundaries of the hydrophobic barrier. Therefore, the minor variations in the initial location of the pipetting step are negated and the precise location of the culture is known for further automated steps. In particular, this confers an advantage to the automated visualisation of the culture.

The hydrophobic barrier may be made of any material which is capable of preventing the movement of the liquid culture across a porous membrane and, thus, retaining the cell culture. In one embodiment the hydrophobic barrier is made of a hydrophobic ink. The hydrophobic ink can be drawn onto the porous membrane in the desired shape and size, or more usually will be printed onto the membrane with the desired size and shape. Examples of such inks include carbon commonly used as an ink source for laser printers and photocopiers, silicone inks and acrylic inks.

In an alternative embodiment the hydrophobic barrier is a laminated layer which is pre-shaped before application to the porous membrane. The laminated layer is a sheet of hydrophobic material for example plastic polymers from which one or more sections have been removed. The removal of one or more sections from the laminate layer creates one or more voids. The void may be circular, dumbbell shaped or any other shape depending on the shape of the hydrophobic barrier required. The laminated layer is applied to the porous membrane such that the edges of the void in the laminate layer act as the hydrophobic barrier when the cell culture is placed on the porous membrane within the void area. Preferably, the laminated layer will be fused to the membrane by gluing, by heat-sealing or by ultra-sonic sealing.

During cell culture, the culture is maintained on the surface of the porous membrane that is disposed at one end of the conduit. One key feature of the device is that the conduit is designed such that, during cell culture, the force of capillarity maintains contact between the surface of the porous membrane contralateral to the cell culture, i.e. the surface of the membrane in the conduit, and the culture medium.

The use of the force of capillarity to maintain the culture medium in the conduit enables the removal and replacement of the culture medium by a pipetting step. When supplying the medium, the pipette tip should be positioned as closely as practicable to the surface of the membrane.

Preferably, the conduit is adapted such that it retains a sufficient volume of liquid culture medium by capillarity to maintain contact between the surface of the porous membrane in the conduit and the culture medium when the device is in either the upright or inverted position. Said conduit may be referred to herein as the medium conduit.

By upright position is meant that the frame holds the conduit substantially vertically with the end sealed by the porous membrane positioned uppermost so that, when the device is in use, the cell culture is grown on the upper surface of the membrane. By inverted position is meant that the frame holds the conduit substantially vertically with the open end positioned uppermost and the end closed by the porous membrane lowermost so that, when the device is in use, the cell culture in the lower surface of the membrane. In contrast to the devices that are available in the art, the device of the invention thus allows incubation of the cell culture and change of the medium for the cell culture with the device in either the upright or inverted position. This flexibility in orientation of the culture and the device means that either microscopes with their objective lenses facing upwards or microscopes with their objective lenses facing downwards can be used interchangeably for studying the culture, and that liquid handling devices can be used in either orientation to add or remove the medium.

Preferably, the conduit is a cylinder, a cone or is frustoconical. Where the medium conduit is a cone the porous membrane is sealed across the narrowest radius of the cone. The conduit may also be of rectangular or asymmetrical cross-section. The exact dimensions and composition of the conduit are selected such that, during cell culture, it retains a sufficient volume of liquid culture medium by capillarity to maintain contact between the surface of the porous membrane in the conduit and the culture medium, preferably irrespective of whether the device is in the upright or inverted position. The volume of liquid retained should be sufficient such that in use, adequate nutrients are supplied to the cell culture without requiring the medium to be changed at unreasonably short intervals.

Capillarity is dependent on several parameters. The force of capillarity is an inverse function of the diameter of a cylindrical vessel or the width or breadth of a conduit of rectangular section. The force of capillarity on an aqueous solution also depends on the surface tension of the solution being held by that force which can be weakened by the presence in solution of surfactants such as detergents. Capillarity is affected by the degree of attraction between the molecules of the liquid and the molecules of the surface. In the case of an aqueous liquid, capillarity is affected by the degree of hydrophilicity of the surface of the conduit. A further factor affecting the retention of liquid culture medium in a conduit is the volume of the culture medium. These factors therefore need to be taken into account to ensure that the device of the invention can retain a volume of liquid media in contact with the surface of the porous membrane by capillarity.

In the device of the present invention, two different capillary forces act to retain the liquid medium in the conduit in contact with the porous membrane. The force of capillarity exerted by attraction between the liquid medium and the tube is one force. The other force is exerted by attraction between the liquid medium and the walls in the pores of the membrane. If sufficiently strong, the former will counteract gravity to keep the liquid in the conduit irrespective of whether it is upright or inverted, and the latter will keep the liquid in contact with the membrane. At a certain threshold, the force of gravity on the culture medium will exceed the force of capillarity and culture medium not restrained by an additional force will fall from the conduit.

Where the conduit is a cylinder, the mass of the liquid contained in the cylinder and thus the gravitational force acting to remove the liquid from the cylinder is directly proportional to the square of the radius of the cylinder, whereas the capillary force acting to retain the liquid in the cylinder is inversely proportional to the radius. Thus for a given liquid and cylinder length there is a maximum radius above which the liquid in a cylinder of a given surface composition will not be retained against the force of gravity, but there is no minimum radius below which liquid will not be retained against the force of gravity.

Preferably, the conduit is a cylinder having a radius of 0.5 cm or less, preferably 0.3 cm or less, preferably 0.25 cm 0.2 cm, 0.15 cm or less or is a cone having a maximum radius of 0.8 cm. Preferably, the cylinder has a radius of approximately 0.3 cm, 0.15 cm or 0.075 cm. It has been found that cylindrical conduits having a radius of 0.5 cm or less or cones having a maximum radius of 0.5 cm or less are adapted to maintain a 1 cm column of a standard liquid culture medium, such as Dulbecco's Minimum Essential Medium, in contact with the surface of the porous membrane in the conduit, irrespective of whether the device is in an upright or inverted position. Preferably, the conduit, preferably a cylinder or cone, is about 1 cm in length, to allow it to retain a 1 cm column of liquid. Preferably, the conduit is slightly greater than 1 cm in length, preferably approximately 1.1 cm or 1.2 cm in length.

Preferably, the conduit is made of a hydrophilic material, preferably a hydrophilic polymer, to increase the force of capillarity exerted on the liquid medium when it is in the conduit. Hydrophilic polymers will be known to the person skilled in the art. The hydrophilicity of polymers from which the conduit is made may be increased further, for example by inclusion of polyethylene glycol groups.

The invention is not limited to cylinders or cones with a maximum radius of less than 0.5 cm as it will be well within the skilled person's ability to determine the dimensions of other conduits which may be used in the device. Specifically, the skilled person will be able to calculate the forces of capillarity and gravity exerted on a given volume of liquid culture medium in conduits of different dimensions and thus determine what dimension of conduit should be employed in the device to ensure that the forces of capillarity exceed the forces of gravity such that the liquid is retained in the conduit. Furthermore, constrictions, platforms or other obstructions may be included in the conduit to increase resistance to the force of gravity acting to remove the medium from the conduit.

According to the Laplace-kelvin equation,

force of capillarity=surface tension/(R1−R2),

where R1=the radius of the tube or pore (in this case the conduit) in cm and R2=the thickness of the meniscus layer in contact with the wall of the tube or pore.

1 dyne is the force required to accelerate 1 gram at 1 cm sec⁻². The surface tension of an aqueous medium is about 73 dyne cm⁻² unless surfactants such as detergents are included. It is not common practice to include detergents in culture media but proteins can also affect surface tension and proteins are commonly included in media particularly in the form of serum. Generally, the surface tension of a liquid culture medium is at least 50 dyne cm⁻². The total force of gravity acting on a given volume of liquid culture medium is 98×(volume in cm³) dyne. The thickness of the meniscus layer (R2) generally need not be taken into consideration when calculating capillarity for the purpose of the present invention. When R2 is small, it has a negligible effect on capillarity and as R2 approaches R1, the capillary force becomes greater. As it is only necessary to determine whether the minimum capillary force requirements are met for a given conduit and aqueous medium for the purpose of the present invention, measurement of R2 is not therefore necessary. It is, however, of course possible to measure R2 if it is desired to calculate the force of capillarity more precisely.

For a cylinder of length 1 cm and a radius of 0.5 cm, a total capillary force of at least 77 dyne would therefore be required to counteract the force of gravity and maintain a 1 cm column of liquid with surface tension 50 dyne cm⁻² in the cylinder by capillarity when inverted. If the hydrophilicity of the cylinder surface is sufficiently high, the force of capillarity can apply a force of greater than 100 dyne to such a column of liquid.

For a cylinder of length 1 cm and a radius of 0.3 cm, a total capillary force of at least 28 dyne would be required to counteract the force of gravity and maintain a 1 cm column of liquid with surface tension 50 dyne cm⁻² in the cylinder when inverted. If the hydrophilicity of the cylinder surface is sufficiently high, the force of capillarity can apply a force of greater than 170 dyne to such a column of liquid.

These forces of capillarity are sufficient to retain such a column of liquid when inverted provided that the device is not moved or vibrated, because accelerations caused by movement or vibration change the momentum of the column of liquid and can overcome the restraining force. Preferably, the dimensions of the conduit are such that no reasonable changes in momentum such as may be caused by normal manual or robotic manipulations result in the loss of liquid from the conduit.

Preferably, the dimensions of the conduit are selected such that the capillary force acting to retain a given volume of liquid medium at the surface of the porous membrane is at least 6 times the gravitational force acting to release the medium. A capillary force of 6 times the gravitational force has been found to be adequate to ensure retention of liquid media in the conduit of the device under normal handling, even when the medium contains protein components such as those in serum that diminish the surface tension of the medium.

Preferably, the porous membrane is fused across one end of the conduit by gluing, by heat-sealing or by ultra-sonic sealing. The porous membrane applies a capillary force to the liquid in the conduit according to the Laplace-Kelvin equation (see above), depending on the radius and surface composition of the pores in the membrane. This capillary force exerted by the membrane should be sufficient to wet the membrane and keep the liquid in contact with the membrane.

Preferably, the porous membrane in the device of the invention comprises pores with a size of ˜0.4 μm. Membranes suitable for use in the device of the invention include but are not limited to the hydrophilic polytetrafluoroethylene (PTFE, also known under the DuPont trade name Teflon®) membrane produced by Millipore Corporation which is optically transparent, membranes made of polycarbonate, PET (polyethylene terephthalate), or Anopore™ (inorganic aluminium oxide, a trademark of Whatman Corp).

Preferably, the porous membrane is optically transparent. This feature enables the test cultures to be accessible at all times to microscopic examination and sampling for biochemical assays. Preferably, the porous membrane produces low background fluorescence at the wavelengths used for excitation, usually in the range of 400-750 nm. Preferably, the porous membrane is composed of hydrophilic polytetrafluoroethylene (PTFE) membrane.

The culture device of the invention may further incorporate one or more electrode for the measurement of electrophysiological response in the cell cultures produced. The electrode(s) may be located in the membrane, below the membrane, above the membrane, or in a combination of any of these locations.

Furthermore, the culture device of the invention may further incorporate one or more electrodes for the stimulation of the cell cultures produced. For example, cardiomyocyte cells cultures may be stimulated with an electric current from the electrodes. As with the electrodes for measuring an electrophysiological response, the electrode(s) may be located in the membrane, below the membrane, above the membrane, or in a combination of any of these locations.

Examples of membranes containing electrodes are known, for example from European patent EP1133691.

The electrodes may be located in the membrane within the area defined by the hydrophobic barrier. The use of the device of the invention in combination with electrodes for the measurement of electrophysiological response or for stimulating the cell culture is advantageous as it concentrates the cells being studied into a specific area thereby allowing improved electrophysiological measurements to be taken from the cells or improved stimulation of the cells. In addition, as discussed above, the hydrophobic barrier allows the precise location of the cells to be known, and therefore the electrodes can be located more accurately in contact with the cells.

Preferably, the frame holds the conduit in a vertical orientation such that neither the end of the conduit closed by the membrane nor the open end of the conduit is in contact with any surface. Preferably, the device further comprises a sealing ring which ensures that the frame is held firmly in contact with the conduit. Preferably, the device comprises two such sealing rings. The device may further comprise additional means to ensure that the frame is held firmly in contact with the conduit so that the conduit is not released when it is inverted. Such additional means may comprise, for example, friction means such as springs between the frame and the conduit.

Preferably, the device further comprises a chamber enclosing the open end of the conduit. The chamber may form part of the frame holding the conduit in a vertical orientation. When the device is in use, the chamber contains an atmosphere of suitable gaseous composition that contacts the medium in the conduit to maintain optimum acidity and oxygen levels in the medium. The chamber is preferably sealed to ensure that the liquid medium is not exposed to the external atmosphere during use. The chamber may further comprise a gas inlet and a gas outlet to allow control of the atmospheric conditions in the chamber.

Preferably, the sealed chamber further comprises an opening to allow the culture medium to be changed. Preferably the opening is designed to minimise exposure of the culture medium to the atmosphere when the medium is changed. The opening may be sealed by a septum or valve that it is normally sealed but may be penetrated by a pipette tip to withdraw the medium and introduce new medium. The septum may be made of rubber or neoprene. The opening may also be used to introduce specific components to the existing medium, such as growth factors or antibiotics or toxins, rather than to change the medium completely. Preferably, the pipetting step is conducted without subjecting the culture to a significant change in hydrostatic pressure.

It will be apparent to those skilled in the art of manual and robotic pipette construction that to withdraw liquid from the conduit, a negative pressure must be applied that is greater than the pressure retaining the liquid in the conduit. It will be important to avoid damage by the pipette tip to the porous membrane, and for this reason the pipette tip will not be advanced into contact with the said membrane. It may not therefore be possible to remove all of the liquid medium from a conduit with a single pipetting step. Liquid may be retained in the conduit in the region of the conduit between the point of furthest travel by the pipette tip and the membrane. Such retention of liquid in the conduit by capillary force is most likely to apply with very small cylinder radius, although it will also depend upon the precise properties of the pipette tip and the liquid. If retention of liquid does occur, it will not, in most cases, affect the health of the culture.

In some circumstances, however, for example if exposure of the culture to a toxic substance is being tested, retention of liquid could potentially influence experimental data. In this case the pipetting steps of liquid removal and replacement with fresh liquid may be repeated as many times as necessary to remove the toxic substance by dilution. For example, if the cylinder is 1 cm long and the pipette tip can be safely advanced to within 0.1 cm of the membrane, then at most 10% of the volume may be retained in the cylinder. The addition of fresh liquid to the full 1 cm length would dilute the toxin to 10% of its original concentration. Repetition of this process would dilute the toxin to 1% of its original concentration. The time programming of pipetting steps would take into account the need to allow equilibration of the toxin to maximise the efficiency of removal by dilution.

Preferably, the device further comprises a lid that covers the surface of the porous membrane outside the conduit. The lid covers the surface of the porous membrane on which the culture is located when the device is in use. Where the device comprises a lid, the chamber and the frame preferably comprise additional ports to allow gas flow between the chamber and space above the membrane enclosed by the lid, allowing the atmosphere surrounding the culture to be controlled over periods of several weeks or more.

The device of the first aspect of the invention is preferably adapted for use in high-throughput methods that involve preparing and maintaining multiple cell cultures simultaneously. According to a second aspect of the invention, there is therefore provided a device for high-throughput cell culture comprising multiple devices according to the first aspect of the invention. Preferably, the device for high-throughput cell culture comprises 96, 384, 1536 or more devices according to the first aspect of the invention.

The device of the second aspect of the invention may thus contain thousands of medium conduits, and each medium conduit can be supplied independently with culture medium and for which the culture medium can be changed independently. Preferably, the medium change is carried out by a multichannel pipette or robot as described above.

Preferably, the high-throughput device comprises a single lid covering all of the individual conduits within the device.

Preferably, the chambers enclosing the open ends of each medium conduit in the high-throughput device are connected by an opening, allowing gas flow between the chambers so that gas flow to all of the chambers within the device may be controlled by a single gas flow inlet and outlet in the high-throughput device.

The multiple devices in the high-throughput device may be fabricated as a single unit. Alternatively, the high-throughput device may be supplied as individual devices according to the first aspect of the invention each containing a single medium conduit that can be assembled into a high-throughput device containing the desired number of conduits by the user. The high-throughput device may also be supplied as strips of individual devices according to the first aspect of the invention, for example, in batches of 2, 4, 8, or 12 that can be assembled into a high-throughput device containing the desired number of conduits, optionally by the user. High-throughput devices comprising strips containing a set number of wells are known in the art for cell culture, although they do not confer the advantages that the device of the invention does. A multiwell device of this type has been described by Dynatech in Thorne A. (1979) in U.S. Pat. No. 4,154,795.

Preferably, for high-throughput devices, the overall size of the device and the position of the individual conduits within the device should match the size of a standard microtitre plate to enable the device to be use with robotics designed for standard microtitre plates. For example, in a high-throughput device comprising 96 devices according to the first aspect of the invention, the devices are preferably arranged in an array of 8 by 12 devices, resembling a standard 96 well microtitre plate. The conduits in the 96 devices making up the high-throughput device are preferably cylinders. Preferably each cylinder or cone comprising a medium conduit has a radius of approximately 0.3 cm which is the radius of a well in a standard 96 well microtitre plate. The capillary and gravitational forces acting in such a cylinder have been described above.

In a high throughput device comprising 384 devices according to the first aspect of the invention, the medium conduit in each device is preferably a cylinder or cone and the cylinder or cone radius is preferably approximately 0.15 cm, the radius of a well in a standard 384 well microtitre plate. The weight of the liquid in this cylinder or cone of the same 1 cm length is only 25% of the corresponding weight with a cylinder or cone diameter of 0.3 cm, but the capillary force is doubled compared to the aforesaid larger cylinder or cone. In a high throughput device comprising 1536 devices according to the first aspect of the invention, the medium conduit in each device is preferably a cylinder or cone and the cylinder or cone radius is preferably approximately 0.075 cm, the radius of a well in a standard 1536 well microtitre plate. In this case the weight of liquid in the cylinder or cone of the same 1 cm length is only 6.25% of the corresponding weight with a cylinder or cone diameter of 0.3 cm, but the capillary force is four-fold higher. Thus, devices of 96, 384 or 1536 medium conduits made according to the invention to the overall size of a standard microtitre plate all retain liquid in the medium conduits in the inverted position.

According to a third aspect of the invention there is provided a method for culturing cells using a device of either the first or second aspect of the invention. The method comprises, incubating a cell culture on the surface of the porous membrane contralateral to the surface of the porous membrane sealed to the medium conduit, within the hydrophobic barrier.

The cells used in the cell culture methods of the invention may be primary cells, embryonic stem cells, adult stem cells, or progenitor cells

### Stem Cells

In one embodiment of the methods of the invention the cell culture is a stem cell culture. By “stem cell” is meant a multipotent cell. The term “stem cell” includes “embryonic stem cells”, “adult stem cells”, “progenitor cells” and “induced pluripotent cells”.

By “embryonic stem cells” is meant a pluripotent stem cell capable of differentiating into the three somatic germ layers that comprise an organism: mesoderm (muscle, bone, etc), ectoderm (neurons, skin, etc) and endoderm (hepatocytes, pancreatic beta cells, etc).

By “adult stem cells” is meant a stem cell which is found in different tissues of the developed, adult organism which remains in an undifferentiated, or unspecialized form. These stem cells can give rise to specialized cell types of the tissue from which they came, i.e., a neural stem cell can give rise to a functional nervous tissue-like parenchyma comprising the different cell types (neuronal and glial cells). The degree of self renewal and differentiation potential of adult stem cells is more restricted when compared to embryonic stem cells. Adult stem cells are multipotent, not pluripotent.

By “progenitor cells” is meant a multipotent cell which can differentiate only into cells of one tissue or germ layer. A progenitor cell is an early descendant of a stem cell that can only differentiate, but can only partially renew itself for a determined period of time.

By “induced pluripotent stem cell (iPS)” is meant a type of pluripotent stem cell artificially derived from a non-pluripotent cell, typically an adult somatic cell, by inducing a “forced” expression of certain genes. iPS cells are believed to be identical to natural pluripotent stem cells, such as embryonic stem cells in many respects, such as the expression of certain stem cell genes and proteins, chromatin methylation patterns, doubling time, embryoid body formation, teratoma formation, viable chimera formation, and potency and differentiability.

The methods of the invention include culturing any of the known types of stem cells, including embryonic stem cells, adult stem cells, induced pluripotent stem cells (iPS cells) from adult somatic cells and progenitor cells.

### Primary Cells

In another embodiment, the methods of the invention are suitable for culturing primary cells. By “primary cells” is meant that the cells are fully differentiated and specialised into a particular cell type. For example cells taken from the central nervous system, blood (e.g. monocytes), spleen, thymus, heart, mammary glands, liver, pancreas, thyroid, skeletal muscle, kidney, lung, intestine, ovary, bladder, testis, uterus or connective tissue.

Primary cell cultures may be formed from dissociated cells or microexplants taken from organs. As used herein, the term “dissociated cell” refers to a single cell that has been isolated from an organ. The term “microexplant” refers to a small group from 400 cells to up to few thousands cells isolated from the organ. Where the method of the invention refers to primary cells the culture comprises of more than one dissociated cell, or of more than one microexplant. Preferably, the method of the invention involves the culture of many dissociated cells, or many microexplants, isolated from an organ.

Where the methods of the invention relate to culturing primary cell, the methods further include the preliminary step of isolating the cells from the organ.

Methods for isolating dissociated cells from organs are known in the art. The dissociated cells may be isolated from the organ of interest by mechanical or enzymatic dissociation of tissue, or both. For example the dissociated cells may be obtained by dissociation of the organ using the proteolytic enzyme trypsin 0.25% (w/w) in Hank's Balanced Salt Solution (HBSS) without calcium and magnesium. After the addition of trypsin inhibitor to stop the enzymatic dissociation, the cells may be incubated briefly in suspension to allow undissociated cells to fall to the bottom, leaving the dissociated cells in suspension.

The microexplants and explants used in the methods of the invention may be obtained by mechanical reduction of the organ of interest to small pieces of tissue. For example, the microexplants may obtained by repeated aspiration, usually of post-natal tissue, in a disposable pipette tip, or by maceration with a scalpel blade. Preferably, the tissue is neonatal tissue.

The methods of the invention may be used to produce an organotypic culture from a wide variety of organs and the nature of the cells that are used in the process will depend on the organotypic culture that is desired. Preferably, the organ from which the cells are obtained is an animal organ, preferably a mammalian organ, preferably a human organ.

The cells may be obtained from any organ in the animal including, but not limited to the central nervous system, bone marrow, blood (e.g. monocytes), spleen, thymus heart, mammary glands, liver, pancreas, thyroid, skeletal muscle, kidney, lung, intestine, ovary, bladder, testis, uterus or connective tissue. Preferably, the dissociated cells, explants or microexplants are from the central nervous system, heart, liver or kidney. Where the dissociated cells, explants or microexplants are from the central nervous system, they may be from the brain or from the spinal cord. Preferably, the cells are from the brain, preferably from the hippocampus or the cortex.

The cells may be obtained from a particular region of the organ. For example, where the organ is brain, the cells may be obtained from the hippocampus or from the cortex. As demonstrated in the examples herein, dissociated cells from the cortical region can be used to produce an organotypic culture that shows the typical cell composition and intercellular connections of hippocampus. Where the organ is heart, the cells may be obtained from the myocardium.

The cells may be obtained from more than one organ and cultured together. For example, the cells may be derived from two, three, four or more different organs. The co-culture of cells obtained from more than one organ allows the generation of models of interactions of tissues derived from different organs. Preferably, where cells from more than one organ are used, the organs will be organs that naturally exist in contact in vivo so that the organotypic culture resulting from co-culture of cells from these organs will provide a model for the in vivo situation. For example, immune cells, particularly white blood cells, could be co-cultured with cells from various organs to study inflammation. Tumor cells might also be co-cultured with cells from various organs to study cancer development. Stem cells could be co-cultured with other cell types to produce mixed cultures. Skeletal muscle cells could be co-cultured with cells from the central nervous system, including hippocampus, cortex, cerebellum and spinal cord, to produce a model of a neuro-muscular junction. Endothelial cells that line blood vessels could be co-cultured with brain cells to form a model of the blood-brain barrier.

The cells used in the methods of the invention may be derived from healthy organisms or from diseased organism. The ability of the methods of the of the invention to generate aggregates of cells quickly and easily means that the methods will have extensive applications in the production of cell cultures for the study of disease links and for drug screening. Comparison of aggregates of cells obtained by the methods of the invention from healthy organisms and diseased organisms will further current knowledge of disease states and allow the identification of biomarkers and drug targets which are indicative of disease states.

The cells used in the methods of the invention may be genetically altered. For example, the cells may be genetically altered to modulate expression of a drug target or a biomarker. A biomarker is a molecular marker, the presence of which at a certain level or in a certain molecular form indicates the presence of a diseased state. A drug target is a molecular species that can be modulated to affect a disease process, i.e. a molecule through which a drug acts. Changing the nature or level of function of the drug target must have a positive impact on disease outcome, and the target should be of a molecular type that is amenable to modulation. In many cases, information about drug targets is obtained from genetic and other biological studies, and classes of compounds that are known to interact with those targets are available. It is often desirable to modulate the levels of these biomarkers and drug targets in biological systems, and to study the biological consequences.

Alternatively, the cells may be genetically altered to express a visual marker, such as a fluorescent marker, that allows the cells to be tracked visually.

Technologies to express cloned genes and to ablate the expression of cloned or endogenous genes are known in the art. These technologies may be used to increase or decrease expression of a marker, such as a drug target or biomarker, in the cells used in the methods of the invention.

Techniques to increase expression of a cloned or endogenous gene are based on the introduction of heterologous DNA in a form which recruits the cellular expression system, and many different approaches are well known to those skilled in the art. In some cases naked DNA may be used with a lipophilic transfection reagent, the DNA including a strong promoter co-linear with the gene to be expressed and a replication origin that enables cytoplasmic replication of the introduced DNA. In other cases a viral vector may be used to increase the efficiency of DNA introduction. Similarly, means to ablate gene expression that are well known to those skilled in the art including antisense DNA oligonucleotides, peptide nucleic acid and double-stranded RNA interference. In some cases, naked nucleic acid may be used. In other cases, especially for the use of small interfering RNA, expression vectors may be used to express the molecule in a self-assembling hairpin form. It has also been shown that proteins can be introduced directly into cells provided that they are attached to an entity that encourages transport from the exterior to the interior of the cell. The Tat protein of human immunodeficiency virus (HIV) is one such entity, and proteins to be transferred may be produced as fusion proteins with HIV-Tat and introduced into cells (Becker-Hapak M. et al, 2001).

It will also be clear to those skilled in the art that, instead of transforming or transfecting the cells as described above, the cells used in the method of the invention may be from a transgenic animal. For example, the cells may be from a transgenic animal expressing a visual marker, such as a fluorescent marker, of from a transgenic animal in which expression of a particular drug target or biomarker has been increased or decreased.

The cells used in the methods of the invention may be derived from healthy organisms or from diseased organism.

The ability of the methods of the of the invention to generate cell cultures quickly and easily means that the methods will have extensive applications in the production of cell cultures for the study of disease links and for drug screening. Furthermore, the methods of the invention will have extensive applications'for the study of stem cells. In particular, the methods of the invention allow for the screening of compounds which promote differentiation of stem cells into different cell types.

Comparison of cell cultures obtained by the methods of the invention from healthy organisms and diseased organisms will further current knowledge of disease states and allow the identification of biomarkers and drug targets which are indicative of disease states.

As described above, biomarkers are molecular markers which at a certain level or in a certain molecular form indicate the presence of a diseased state. A drug target is a molecular species that can be modulated to affect a disease process. One application of the cell cultures of the invention is in the identification of biomarkers and drug targets.

Screening of several molecular classes, such as proteins and lipids, in cell cultures that express a disease state or the corresponding non-diseased state may be used to identify biomarkers. Validated biomarkers are currently used both to identify carriers of a disease state and to monitor their progress towards normality that may be assisted by a therapeutic regime such as a drug. It is necessary to establish a statistically significant association between a candidate biomarker and a disease state to validate the biomarker for use in clinical trials. The cell cultures of the present invention are ideally suited to biomarker discovery and validation due to the fact that they replicate organ function and physiology and can be generated quickly and easily by the methods of the invention such they are applicable to high throughput assays. The cell cultures of the invention could thus be used much more rapidly and cheaply than whole animals currently used for the identification and validation of biomarkers.

According to a further aspect of the invention, there is therefore provided a method for the identification and validation of biomarkers and drug targets comprising screening the cell cultures produced by the methods of the invention. Assays for identifying biomarkers and drug targets include the use of transcriptional profiling, proteomics, mass spectrometry, gel electrophoresis, gas chromatography and other methods for molecular profiling known to those skilled in the art.

Surrogate markers are a sub-set of biomarkers that can be used to assess the presence or progression of a disease state, but that do not measure directly a clinical outcome of the disease. The cell cultures of the invention may be used to identify and validate surrogate markers in the same way as other biomarkers.

The cell cultures produced by the methods of the invention are not only useful in the identification of biomarkers and drug targets associated with disease states but are also useful in screening to identify drugs that alleviate these disease states. Cell cultures are particularly useful in the screening of candidate drugs because it is important for such screening that the target culture has biochemical and physiological properties that match as closely as possible those features of the target organ in vivo. It must be possible, however, for the cell culture to be used at high throughput to enable screening of sufficiently large numbers of drug candidates for a high probability of successful identification of lead drugs. Additional large-scale assays are often necessary to validate the inclusion of a lead drug in a preclinical and clinical drug development programme.

The methods of the invention may be used to generate many thousands of cell cultures simultaneously and are thus uniquely suited to high throughput applications involving multiple assays for each culture. In one embodiment, the methods for producing a cell culture according to the invention further comprises the step of screening using the resulting cell culture in a method of screening and pre-clinical validation of candidate drugs. As discussed above, one particularly useful aspect of the method of the invention is that it facilitates the high-throughput formation of cell cultures in which the cells have been genetically altered to modulate the expression of a biomarker or drug target. These modified organotypic cultures will also be useful in the screening of candidate drugs.

The field of toxicology is a further application area for the present invention that will benefit greatly by the enhanced flexibility and throughput provided by the methods of the invention. Organotypic response is crucially important in this field, because different tissues differ greatly in their response to toxins, with different clinical consequences. Different tissues can contain different enzymes systems, notably of the cytochrome P450 class, that metabolise different classes of exogenous compounds. The degree and type of metabolism of a compound can profoundly affect its toxicity. Large-scale screening of toxicity in a wide variety of tissues is so expensive at present that many chemicals in common use have never been tested adequately. Increasing awareness of potential toxicity has brought pressure to carry out such tests without the means to do so at acceptable cost. The invention therefore also includes a method of assessing the toxicity of a chemical using the cell cultures of the present invention.

### FIGURES

FIG. 1A: Scheme of the hydrophobic ink printing method on porous membranes. A drop of dissociated cells (1) is deposited onto the membrane that fills the surface delimitated by the ring of hydrophobic ink (7). The culture medium (3) is added in the well (5) and remain there by capillarity and surface tension. B shows a photography of a 96 well format device manufactured following the principle and design illustrated in A. At higher magnification (insert) in C we can observed the formation of the drop (1) constraint by the circular hydrophobic ink (7). The 96 well format device is covered by a lid (9).

FIG. 2: Examples of designs of rings made with black hydrophobic ink used for individual cultures as single spots (A) or dumbbell shapes dedicated to co-culture monotypic cells or cells from different origins as co-spots (B)

FIG. 3: Different volumes of PO cortical dissociated cell solutions were deposited as drops onto PTFE membranes (A: 1 ul; B: 2 ul; C: 3 ul) either without (A,B) or with (C) a 2 mm ring (2 mm internal diameter) of hydrophobic black ink. Pictures A and B show irregular and flat aggregates of cells while a regular dome like structure can be seen using the ring of hydrophobic ink method. By using hydrophobic ink, we can thus control the volume and the cell density of the Hi-Spot which critically affects the evoked responses that can be recorded from the Hi-Spots.

FIG. 4: Picture A shows a PO cortical aggregate that was laid down onto a multi-electrode array. We can take advantage of the constraints induced by the hydrophobic property to generate thicker structures with a lower surface of contact. This phenomenon can be interesting to obtain an important density of neurons that will generate, for example, good extracellular electrophysiological signals as illustrated by the input/output curve showed in B. The amplitude of the evoked field potential signal is depending on the density of cell per square mm that can be achieved by building a thick 3D structure.

FIGS. 5 A and B: scheme of the dumbbell design of hydrophobic ink use to generate 3D co-cultures. Cell bodies (17), axons (19) and a connecting chamber (cells or gels) are shown. FIG. C shows a co-culture of neurons from GFP transduced neural stem cells with Sin-1 promoter to specifically visualize neurons. Neural cells were grown at both extremities where the soma of neurons is located (see C1 and C3) separated by a gap filled with hydrogel or matrigel. Outgrowth axons from neurons from both sides were observed crossing the entire gap to connect neurons from the controlateral side. Note the growth cone in C2 indicated by the arrow (13). Microphotographs were taken using one week old cultures.

FIG. 6A: microphotography of a cortical PO co-culture laid down onto a dedicated multi-electrode array to fit to the dumbbell shape where neuron cell bodies and axons are located. B: extracellular evoked field potentials where recorded by using stimulating electrodes (25) close to the recording electrodes (27) (B1) inducing a signal after only 1 ms or after 6 ms when the stimulating electrodes are located at distance (B2). Electrophysiological recordings were performed on 10 day-old cultures.

FIG. 7A: microphotography of cardiomyocyte cultures derived from human ESCs at low (A1) or high density (B1). When cultures from 5 days up to 2 month old were placed onto multi-electrode arrays, electophysiological signals were recorded at frequencies generally between 0.5 up to 2 Hz (B1). At higher magnification (insert B2), we can clearly see the repolarising potentials (29)

FIG. 8A: Electrophysiological signals recorded before (A1) and after the addition of 30 uM of lidocaine, a sodiun ion channel blocker (A2) B: Kinetic of the amplitude of the signals after the addition of 30 uM of lidocaine (arrow). A 50% decrease of the amplitude can be seen after 6 minutes.

FIG. 9: Electrophysiological signals recorded before (control) and after the addition of 30 nM of the HERG potassium ion channel blocker E4031 after 5 and 15 min. Note the progressive shift of the repolarising potentials from 140 ms in control recordings to 155 ms after 5 min of treatment and finally 173 ms after 15 min in presence of the molecule.

FIG. 10: The bright field micro-photography (X4) in A shows the resulting aggregate from neural stem cells Sin-1-GFP transduced on top of the membrane at the air/liquid interface. In B, the sample was taken at under the fluorescent light at low magnification (X4) and at higher magnification in C(X10) and insert showed in D (X20) where the neuronal cell bodies as well as neurites can be visualized (arrow).

### EXAMPLES

### Example 1

### Cell Culture Hi-Spot Protocols

### 1) Neural Primary Cells

Decapitate Wistar rat pups (New Born P0) and dissect the cortices quickly into cold EBSS supplemented with MK801. (Roughly 3-4 mls of EBSS per animal and 1 μl of 10 mM MK801 per ml of EBSS is needed). Triturate the tissue using flame polished pipettes and pass the suspension through a BD cell strainer to remove debris. Count the cells and centrifuge at 1500 rpm for 5 minutes. Remove any supernatant and re-suspend the cells to 50,000 cells/μl in cortical Media.

Plate 5 μl of cell suspension onto millicell inserts (5 per well) and maintain the cultures at 37 degrees/5% CO₂ for the required duration.

Maintenance medium consists of cortical media (1) for initial 7 days followed by Neurobasal (2) for the remainder. Media is changed twice a week.

CORTICAL MEDIA

| | 500 mls | | Final Conc. | |
|---|---|---|---|---|
| 1. Ham's F12 | 50 | ml | 10% | Sigma 51651C |
| 2. Fetal Bovine | 100 | ml | 20% | Invitrogen |
| Serum | | | | |
| 3. Horse Serum | 25 | ml | 5% | Invitrogen |
| 4. Hepes | 5 | ml | 10 mM | PAA 1M Hepes S11-024 |
| 5. Glutamine | 5 | ml | 2 mM | PAA 200 mM M11-004 |

| | | |
|---|---|---|
| 6. DMEM + glucose | Up to | PAA E15-009 |

CULTURE MEDIUM NEUROBASAL (-A) −2% B27

| | 400 mls | | Final Conc. | | |
|---|---|---|---|---|---|
| 1. Glutamine 200 mM | 4 | ml | 2 | mM | PAA M11-004 |
| 2. B27 Neuromix 50X | 8 | ml | | | PAA FO1-002 |
| 3. Hepes 1M | 0-200 | μl | 0.5 | μM | PAA S11-024 |
| 4. Neurobasal - A | Up to 400 | ml | | | PAA U15-024 |

### 2) Neural Progenitors from ESC D3 Line

Neuronal differentiation of ES cells was induced by co-culture for 7 days with murine bone marrow-derived stromal feeder (MS5) cell line. Purification and propagation of neural precursors cells was then performed by subsequent culture for 2 days in N2 medium supplemented with bFGF (10 ng/ml). At that point, cells were frozen in liquid nitrogen.

Hispot neural culture were prepared from rapidly thawed D3-ES neural precursors cells, washed with N2 medium and plated at high density onto PTFE membrane disks (3 μl, 10'000 cells/μl). The hispots were then cultured for 7 days at interface air/N2 medium added with bFGF (10 ng/ml). Then neuronal differentiation of D3 ES neural precursors was induced by culturing hispots at interface air/MEM plus 25% horse serum, for at least 10 days before experiments were carried out.

### 3) Adult Neural Stem Cells

### TERMS AND ABBREVIATIONS

| | |
|---|---|
| ~ | Approximately |
| bFGF | basic fibroblast growth factor |
| cAMP | adenosine 3′,5′-cyclic monophosphate, N⁶, O^{2′}-dibutyryl-, |
| | sodium salt |
| CO₂ | carbon dioxide |
| DMEM | Dulbecco's modified essential medium |
| EGF | epidermal growth factor |
| ES | embryonic stem cells |
| GDNF | glial derived neurotrophic factor |
| min | Minutes |

Chemicals—Accutase (PAA), B27 supplement (PAA), bFGF (Peprotech), cAMP (Calbiochem), CO₂ (BOC), DMEM (PAA), EGF (Sigma), Ethanol (Calbiochem), F-12 (PAA), GDNF (Calbiochen), Heparin sodium salt (Sigma), Laminin (Invitrogen), L-Glutamine (Invitrogen), Trypan blue (Sigma), Vircon

Materials-1-200 μl, pipette tips (Star labs), 15 mL centrifuge tubes (Greiner), 2-20 μL pipette (Star labs), 10-100 μl, pipette (Star labs), 6 well plate (Greiner), Cell scraper (Greiner), Confetti membrane (BioCell, Switzerland), Fine curved forceps, Haemocytometer (Marienfeld), Incubator (Heraeus), Millicell-CM 0.4 μm culture plate insert 30 mm diameter (Millipore)

P2 Hood (Heraeus), Pipette pump (Star labs), Serological 5, 10 mL pipettes (Greiner), SteriCup filters (Millipore), T75 flask with vented lid (Greiner),

### Method

Incubator settings—37° C., 5% CO₂.

### Preparation of the Flask

cover the surface of the flask with 20 μg/mL laminin in DMEM and incubate for at least 2 hours
wash with sterile DMEM before replacing it with 20 mL of culture medium
keep in the incubator until use

Preparation of the Plate
pipette 1 mL of differentiating medium in each well
transfer the Millicell inserts
place four confetti membranes on each insert
keep in the incubator until use

### Preparation of the Cell Counting

pipette appropriate volume of trypan blue in an Eppendorf reaction tube
place cover slip on an haemocytometer

### Lifting and Splitting the Cells

remove all the medium from the T75 flask containing the confluent RenVM cells
add sufficient Accutase to cover the surface of the cell layer (˜3.5 mL)
incubate for 5-10 min until the cells lift
if they still stick to the plastic scrape them gently using a sterile cell scraper
inoculate new culture (prepared T75 flask from the incubator). Split to a ratio of 1:10
transfer the remaining cell suspension to a 15 mL centrifuge tube
add an equal volume of culture medium to inactivate the Accutase
aseptically remove a sample of the cell suspension to trypan blue for cell counting
centrifuge remaining cell suspension at 1000 rpm for 5 min
remove the supernatant
resuspend the cell pellet in appropriate volume of differentiating medium to give 50,000 cells/μl
pipette 3-5 μL per Hi-spot (50,000 cells/μL on confetti using the 2-20 μL pipette
transfer plate into incubator
change medium twice a week

Calculation of the Amount of Cells in Suspension
count all trypan blue-negative cells in an appropriate number of 1 mm³ squares
calculate the total amount of cells

$$\frac{\begin{matrix}counted cells \times dilution factor \times \\ volume of suspension ( ml ) \times {10}^{4}\end{matrix}}{number of squares counted} = total number of cells$$

calculate the amount of medium needed to re-suspend the cells to 30'000 to 50000/μl

total number of cells=volume needed to resuspend the 30'000 to 50,000 cells

### Media:

| culture | differentiating |
|---|---|
| DMEM:F12 (1:1) | DMEM:F12 (1:1) |
| 1x B27 | 1x B27 |
| 10 U/mL heparin sodium | 10 U/mL heparin sodium |
| 2 mM glutamine | 2 mM glutamine |
| 10 ng/mL bFGF | 1 mM cAMP |
| 20 ng/mL EGF | 2 ng/mL GDNF |

Sterilise the media using a filter with a pore size of 0.02 μm.

### 4) Cardiomyocyte Derived from hESCs

Beating aggregates are maintained at room temperature in cryo-vials full of culture medium (Human cardiovascular progenitor cells develop from a KDR+embryonic-stem-cell-derived population. Yang L, et al., (2008).

Chemicals—CO₂ (BOC), Collagenase IV (Sigma), DNAse I (Sigma), EBSS, w Mg, w Ca, Ethanol (Calbiochem), Trypan blue (Sigma), Trypsin/EDTA, Virkon

Materials-1-200 μL pipette tips (Star labs), 12 μm pore size polycarbonate membranes (Watman), 15 mL centrifuge tubes (Greiner), 2-20 μL pipette (Star labs), 10-100 μL pipette (Star labs), 6 well plate (Greiner), Confetti membrane (BioCell, Switzerland), Fine curved forceps, Haemocytometer (Marienfeld), Incubator (Heraeus), Millicell-CM 0.4 μm culture plate insert 30 mm diameter (Millipore), P2 Hood (Heraeus), Pipette pump (Star labs), Serological 5, 10 mL pipettes (Greiner), SteriCup filters (Millipore)

### Method

Incubator settings 37° C., 5% CO₂.

Beating aggregates are plated on a 6 well plate and left in
the incubator for a few hours
Suspension of EBs are transferred in a 15 mL tube
Centrifuged at 1000 rpm for 1 min
Discharged supernatant
0.2 collagenase

| | | |
|---|---|---|
| 20 ng/mL DNAse | | |
| | {close oversize brace} | Incubation1 h at 37° C.; |
| EBSS | | |

Centrifuged at 1000 rpm for 1 min
Discharged supernatant
Washed with EBSS
Centrifuged at 1000 rpm for 1 min
Discharged supernatant
Incubated in o.o5% trypsin/EDTA at 37° C. for 10 min;
Centrifuged at 1000 rpm for 1 min
Discharged supernatant
Inactivated trypsin with EBSS + 50% FCS + 20 ng/mL DNAse
Centrifuged at 1000 rpm for 1 min
Discharged supernatant completely
+500 μL of culture medium
triturated with a yellow pipette
trypan blue cell counting
plated 3 μL as CardioSpot → ≈30,000 cells/Hi-spot

Culture Medium—StemPro 34 and 10 ng/mL VEGF

### 5) CardioSpot from Rat Primary Cardiomyocytes

Coat confetti in collagen type I overnight and allow to dry in the hood before putting on membranes.
Dissect out rat E16/17 foetus into dissecting solution on ice.
Dissect out whole hearts into EBSS at room temp.
Transfer hearts into EBSS with 2.5 mg/ml trypsin and 200 U Dnase I.
Incubate at 37C for 1 hour.
Wash x3 in EBSS.
Triturate gently using graded flame polished pipettes.
Allow debris to settle to bottom of tube then remove cell suspension into a fresh tube.
Count cells and spin at 2000 rpm for 5 mins.
Resuspend cells in whole heart media to 10'00 to 70,000 cells/μl.
Carefully drop 5 μl of cell suspension into the centre of each confetti.
Change media twice weekly.
Whole heart media
20 mls FCS
30 mls horse serum
37 mls M199
1 ml pen/strep
make up to 200 mls with DMEM

### Example 2

### Electrophysiological Recordings

### 1) Nervous Tissues

Electrophysiological recordings were obtained using a perfusable multielectrode array of 40 electrodes, at 37° C., in a Hepes-buffered extracellular saline solution (HBS) containing in mM: NaCl 140, KCl 1.6, MgCl₂ 1.5, glucose 10, CaCl₂ 2.5, D-glucose 10, Hepes 10 (pH:7.4, adjusted with NaOH).

Eight recording electrodes and 2 stimulating electrodes (200 μs bipolar stimulation) were selected for any given HiSpot. Paired-pulse evoked field potentials were recorded in response to stimulation of typically 2-3000 mV every 30 s., with a paired pulse interval of 30 ms. Data used for input-output curves construction were obtained in response to stimuli ranging from 0 to 4000 mV (one paire-pulse every 5 s.). Spontaneous recordings were typically obtained over 5 minute using the same electrode set as the one used to record evoked activity

### 2) Cardiac Tissues

CardioSpot made using primary or stem cell derived cardiomyocytes are placed onto the porous MEAs. Electrophysiological recordings were performed either through the supporting membrane or from under the membrane in order to get a direct contact of the tissues with recording electrodes. Control recording were performed using the culture medium as well a the solution used for reference molecules (E4031, and lidocaine)

### Example 3

### Results and Conclusion

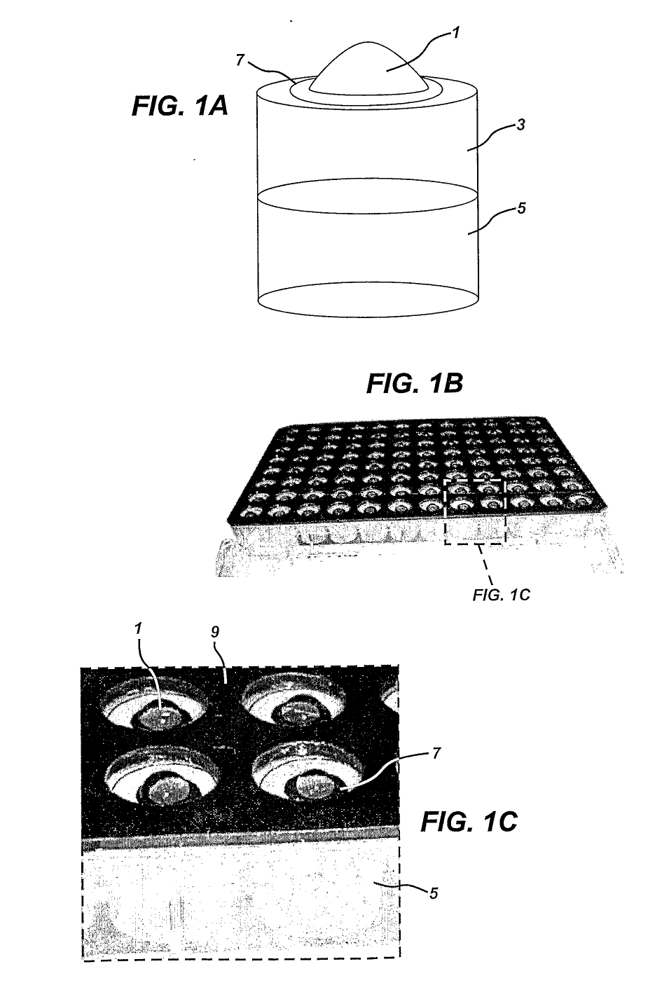

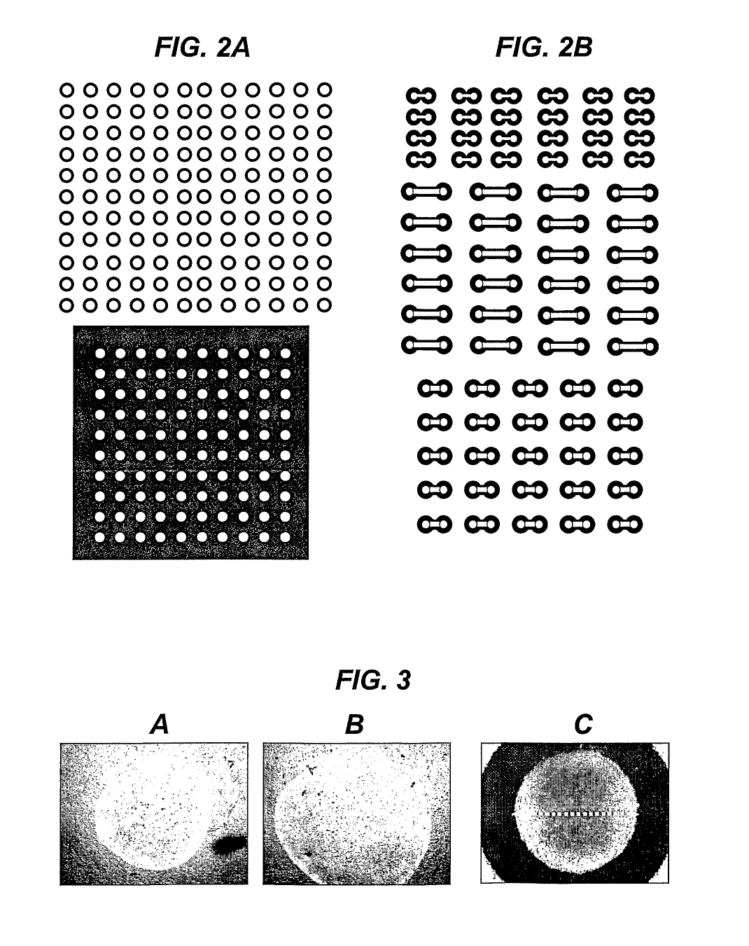

We found that the control of the cell growth within specific designs of hydrophobic barriers, illustrated in FIGS. 1 and 2, allowed us to engineer homogenous three-dimensional structures (FIG. 3) that differentiate and mature overtime with characteristic features similar to those observed in vivo.

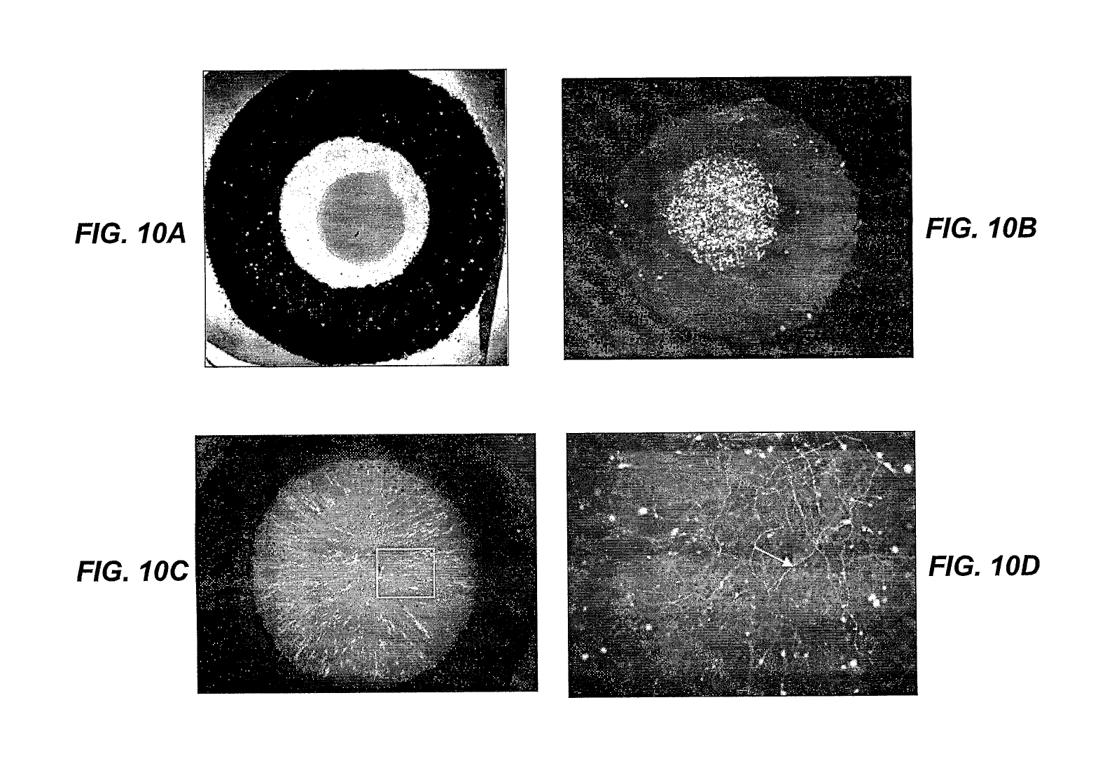

Differentiation and maturation of cell cultures within the hydrophobic barriers will develop tissue-like structures with properties mimicking in vivo situation. For example, when aggregates from neural stem cells Sin-1-GFP are grown on top of the membrane at the air-liquid interface, they differentiate in nervous parenchyma with neuronal cytoarchitecture, formation of neural networks which are similar to native nervous tissues (FIGS. 10C and 10D).

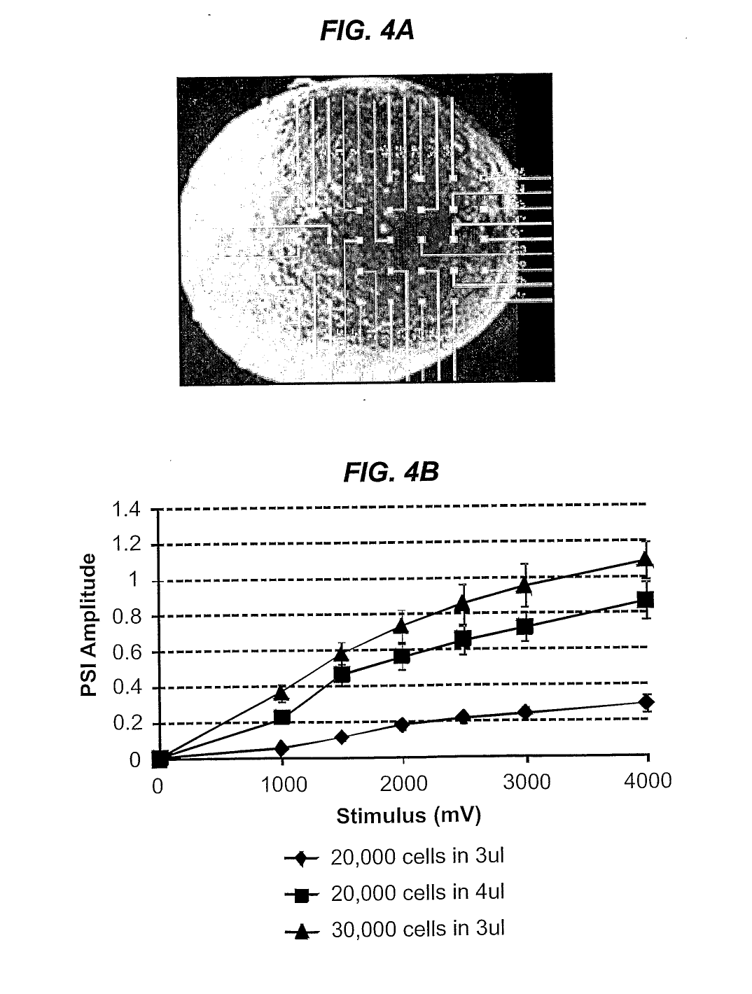

Interestingly, this method enables the use of the minimum of cells needed to recreate functional tissues in vitro. As an example, when nervous tissues are reconstituted either by growing dissociated primary or neural progenitor cells, the input/output curve (obtained by stimulating the tissues with progressive increase of depolarizing voltages) shows that by concentrating a lower number of cells on a smaller surface delimitated by a hydrophobic barrier made of hydrophobic ink, higher amplitudes of evoked field potentials could be recorded (FIG. 4, the line with triangular dots).

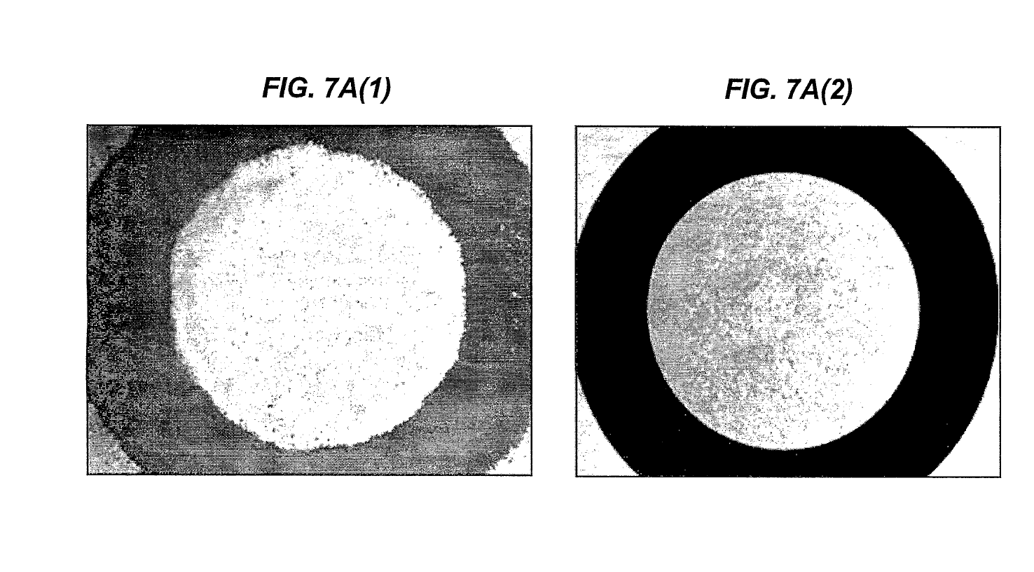

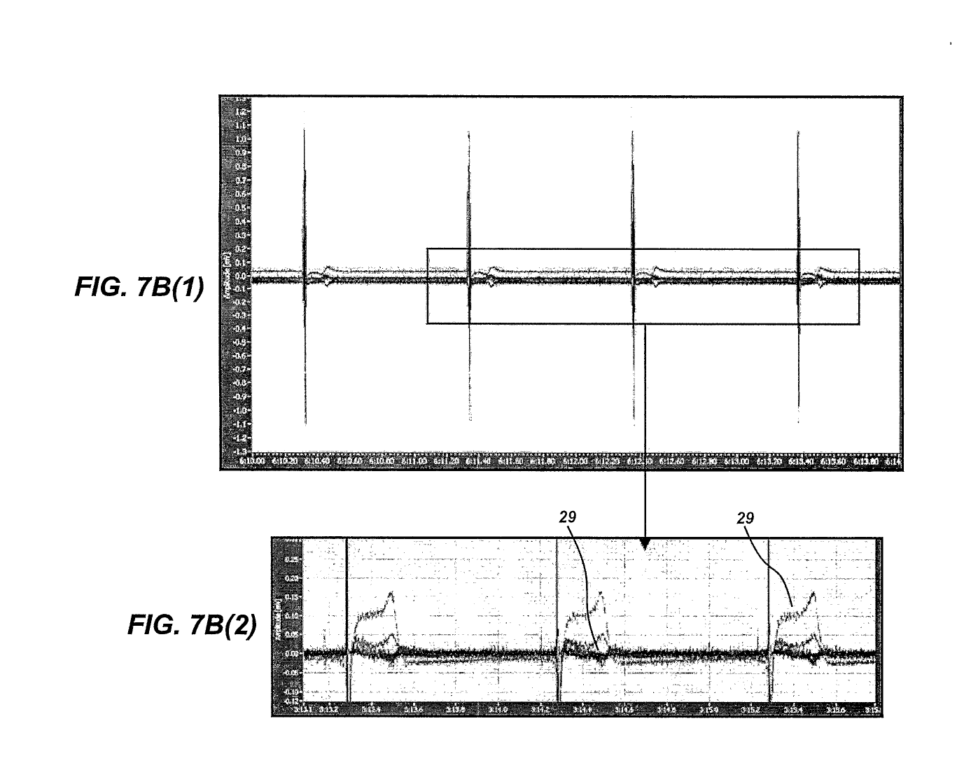

Similar functional results were obtained on heart tissue reconstituted with either primary or derived cardiomyocytes from embryonic stem cells (FIG. 7). Low density of spread cardiomyocytes cultures (FIG. 7A-1) give desynchronised electrophysiological signals whereas, the cultures of high densities of cardiomyocytes on a delimitated surface of membrane forms tissue-like structures (FIG. 7A-2) that beat synchronously with frequencies varying from 0.5 to 2 Hz (FIG. 7B). Rhythmic signal with high amplitudes (0.5 to 5 mV) could be spontaneously recorded when tissues were placed on porous multi-electrode arrays (FIG. 7B-1) as well as repolarising potentials with lower amplitudes 0.05 to 0.15 mV as seen in the insert (FIG. 7B-2).

Pharmacological experiments were carried out using reference compounds to characterise and validate the cardiac tissue generated from cardiomyocytes derived from human ESCs and engineered using the method of invention. For example, we used lidocaine, sodium channel blocker, to verify that the amplitude of the first signal (which is predominantly dependent of sodium channel opening) was effectively diminished. As expected, we could observe a dramatic decrease of the amplitude of the signals within few minutes (FIG. 7).

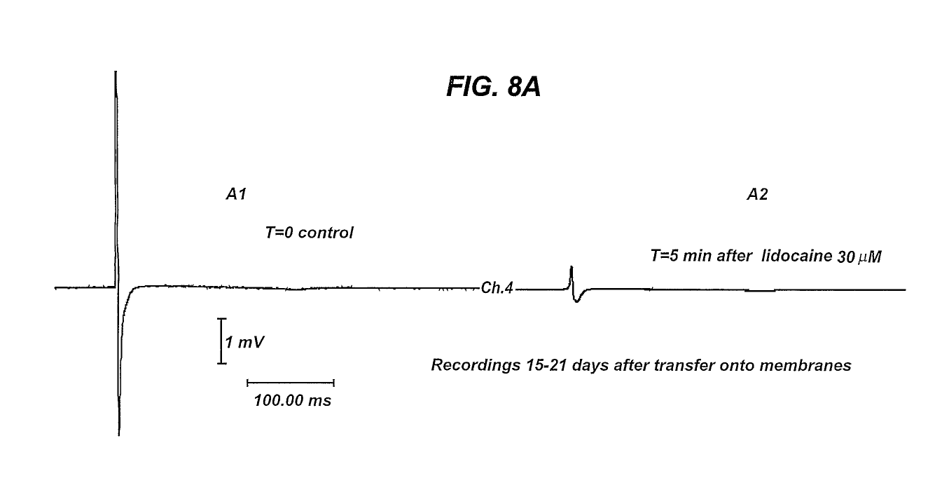

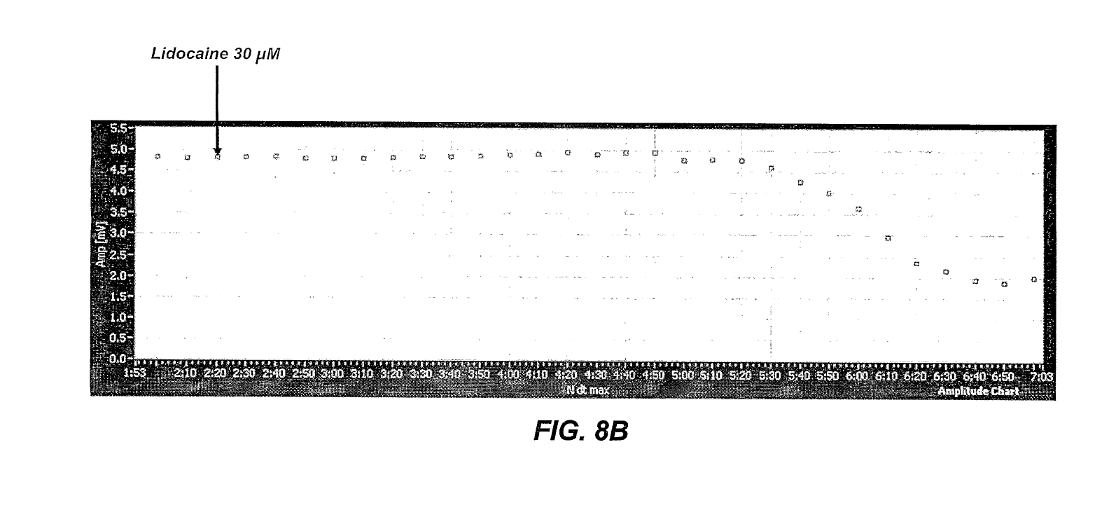

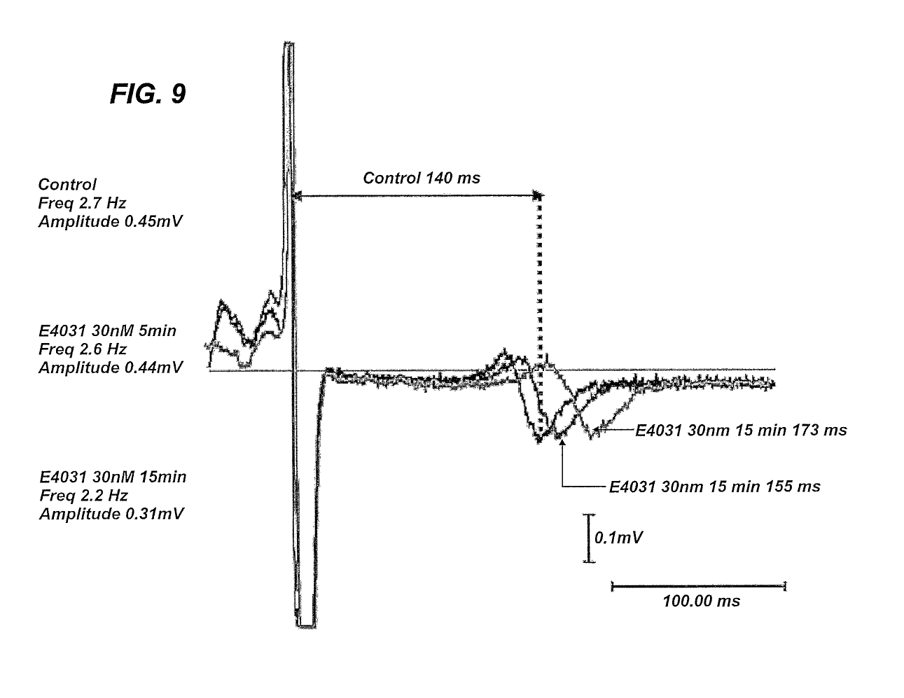

Another reference compound E4031 (a HERG potassium ion channel blocker), that causes a shift in the repolarising potential so that it become delayed (Q-T prolongation) as it does in humans was used in a subsequent series of experiments (FIG. 8). The repolarising potential shifts progressively from 140 ms (delay measured from the first signal) in control medium to 155 ms after 5 min to 173 ms after 15 min of incubation with a concentration of 30 nM of the molecule E4031.

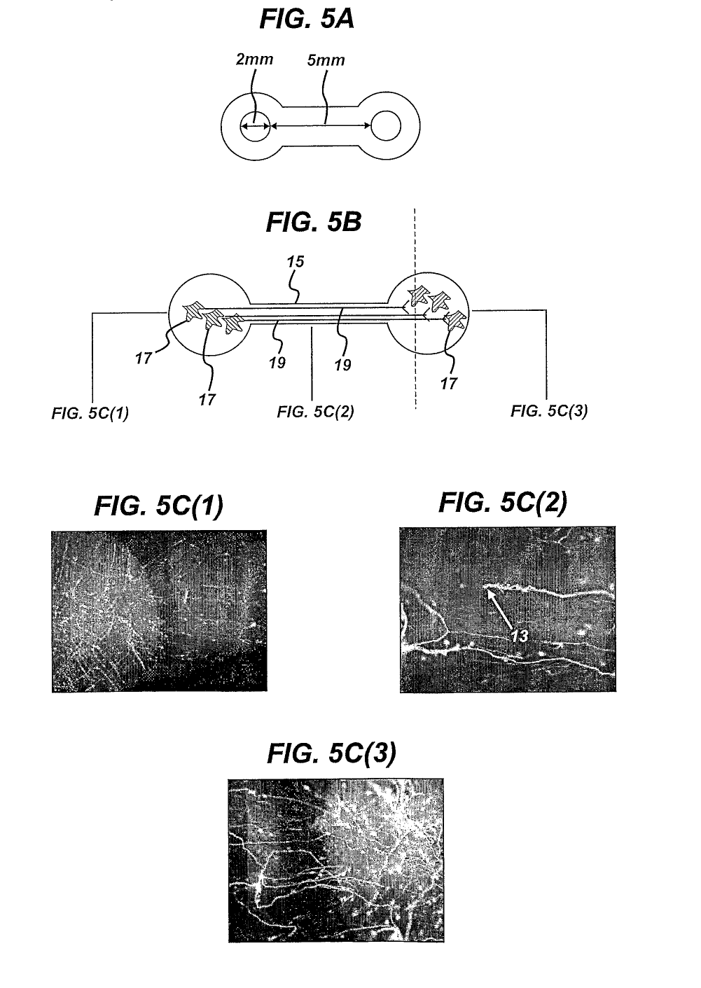

In order to mimic even more closely the in vivo situation, where tissues and organs interact and communicate constantly, a specific design of the invention comprising a dumbbell shaped hydrophobic barrier (FIG. 5A) was tested. The device was shown to promote the relationships between target tissues or different regions within the same organ. We characterized and validated this approach by co-culturing two nervous tissues both placed in the rings of the dumbbell shaped hydrophobic barrier. The gap between the two target tissues was filled either with different types of hydrogel (agarose, matrigel) or cells (scheme of the whole mount, FIG. 5B).

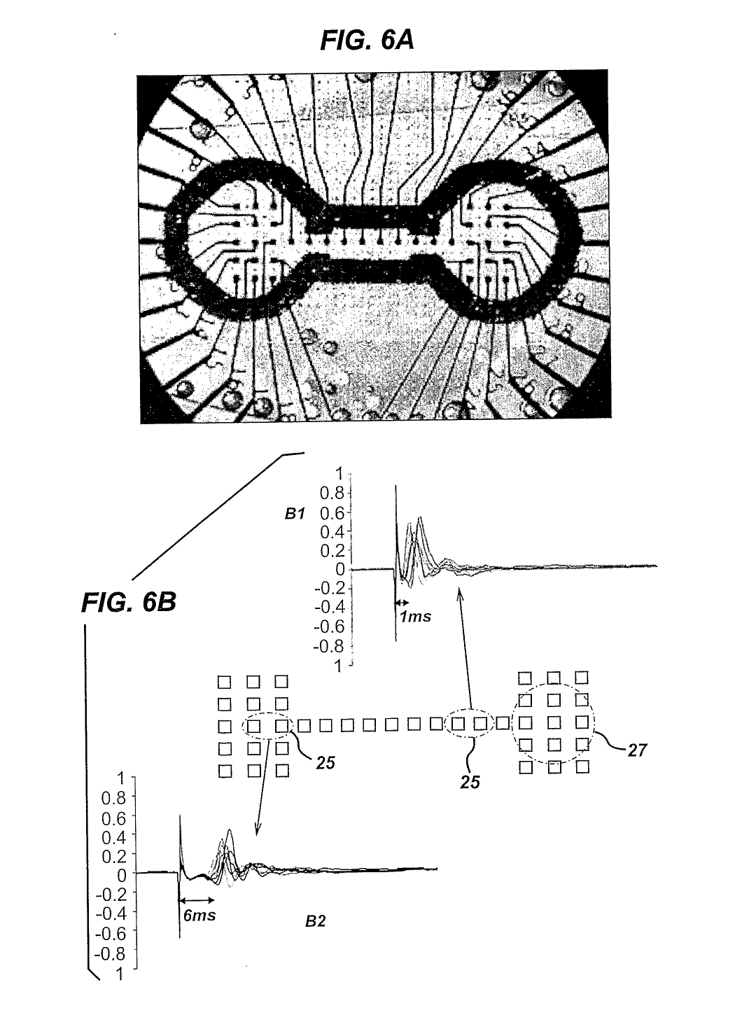

In a series of experiments, neural cells were previously transfected or transduced with specific neuronal promoters with GFP as a tag to visualize neurites (axons and dendrites) as well as neuron cell bodies (FIG. 5C). Outgrowth of axons through the filled gap could be observed after 48 hours and extensions of new fibres were still detected after 10 days in culture (FIG. 5C-2). Functional activities were confirmed by carrying out electrophysiological experiments using a dedicated multi-electrode array design where the different areas of the dumbbell shape can be stimulated and recorded (FIG. 6 A).

In one experiment (FIG. 6 B), electrophysiological recordings were performed in only one tissue whereas stimulating electrodes where located either close to the recording area or on the controlateral side. By varying the distance of the stimulating trigger, we could obtain evoked field responses with shorter (1 ms; FIG. 6B-1) or longer (6 ms; FIG. 6B-2) latency which would be consistent with monosynaptic responses.

The use of a device containing a hydrophobic barrier thus allows the rapid production of reliable organotypic cultures for toxicological screening and drug discovery.

### REFERENCES

Amit, M., Shariki, C., Margulets V. and Itskovitz-Eldor J., Biology of Reproduction 70, 837-845 (2004). Feeder Layer- and Serum-Free Culture of Human Embryonic Stem Cells.
Bentzl Kristine, Molcanyi Marek, Heβ Simone, Schneider Annette, Hescheler Juergen, Neugebauer Edmund and Schaefer Ute, Cell Physiol Biochem 2006; 18:275-286. Neural Differentiation of Embryonic Stem Cells is Induced by Signalling from Non-Neural Niche Cells
Guo X M, Wang C Y, Tian X C, Yang X. Methods Enzymol. 2006; 420:316-38. Engineering cardiac tissue from embryonic stem cells.
Ikeda H, Osakada F, Watanabe K, Mizuseki K, Haraguchi T, Miyoshi H, Kamiya D, Honda Y, Sasai N, Yoshimura N, Takahashi M, Sasai Y. Proc Natl Acad Sci USA. 2005 Aug. 9; 102(32):11331-6. Generation of Rx+/Pax6+ neural retinal precursors from embryonic stem cells.
Sundstrom L, Morrison B 3rd, Bradley M, Pringle A. Organotypic cultures as tools for functional screening in the CNS. Drug Discov Today. (2005); 10(14):993-1000. Review
Sundstrom L E. Thinking inside the box. To cope with an increasing disease burden, drug discovery needs biologically relevant and predictive testing systems. EMBO Rep. (2007); 8:Spec No:S40-3.
Takahashi K, Tanabe K, Ohnuki M, Narita M, Ichisaka T, Tomoda K, Yamanaka S. Induction of pluripotent stem cells from adult human fibroblasts by defined factors. Cell. (2007); 131(5):861-72
Vanderlaan Rachel D., Oudit Gavin Y., Backx Peter H. Circulation Research. 2003; 93:1. Electrophysiological Profiling of Cardiomyocytes in Embryonic Bodies Derived From Human Embryonic Stem Cells.
Yamanaka S. Strategies and new developments in the generation of patient-specific pluripotent stem cells. Cell Stem Cell. (2007); 1(1):39-49
Yang L, Soonpaa M H, Adler E D, Roepke T K, Kattman S J, Kennedy M, Henckaerts E, Bonham K, Abbott G W, Linden R M, Field L J, Keller G M. Nature. 2008 May 22; 453(7194):524-8. Epub 2008 Apr. 23

1. A device for cell culture, said device comprising:
(i) a medium conduit having one open end and one end closed by a porous membrane fused across it; and
(ii) a frame holding the medium conduit in a substantially vertical orientation;
wherein the medium conduit is adapted to permit retention by capillarity of a sufficient volume of liquid culture medium in the medium conduit to contact the surface of the porous membrane and thus supply nutrients to cells that may be grown on the porous membrane, wherein the surface of the membrane contralateral to the surface of said porous membrane sealed to said medium conduit comprises a hydrophobic barrier adapted to contain the culture.

2. The device of claim 1, wherein the medium conduit is adapted to permit retention by capillarity of a sufficient volume of liquid culture medium to contact the surface of the porous membrane in the conduit when the device is in either the upright or inverted position.

3. The device of claim 1, wherein the hydrophobic barrier comprises hydrophobic ink.

4. The device of claim 1, wherein the hydrophobic barrier is circular.

5. The device of claim 1, wherein the hydrophobic barrier is dumbbell shaped.

6. The device of claim 1, wherein the height the hydrophobic barrier projects above the membrane is below about 100 μm.

7. The device of claim 1, wherein the surface of the porous membrane sealed to said medium conduit is adapted to allow electrophysiological measurements.

8. The device of claim 1, wherein the porous membrane is fused across one end of the medium conduit by gluing, by heat-sealing or by ultra-sonic sealing.

9. The device of claim 1, wherein the porous membrane is composed of hydrophilic polytetrafluoroethylene (PTFE).

10. The device of claim 1, comprising a lid that covers the surface of the porous membrane contralateral to the liquid culture medium.

11. A device for high-throughput cell culture comprising multiple devices of claim 1.

12. A device for high-throughput cell culture comprising about 96, 384, 1536 or more devices of claim 1.

13. A method of culturing cells comprising incubating a cell culture using a device of claim 1, wherein the cell culture is incubated on the surface of the porous membrane contralateral to the surface of the porous membrane sealed to the medium conduit, within the hydrophobic barrier.

14. The method of claim 13 further comprises the step of screening the resultant cell culture for the identification and pre-clinical validation of candidate drugs, comprising adding a test compound to the cells and assessing its effect on the proliferation, differentiation and/or phenotype of the cells.
